(19) United States
(12) Patent Application Publication
(10) Pub. No.: US 2014/0243891 A1
(43) Pub. Date: Aug. 28, 2014

(54) TISSUE PROTECTOR SUTURE CONSTRUCTS

(71) Applicant: Arthrex, Inc., Naples, FL (US)

(72) Inventors: Reinhold Schmieding, Naples, FL (US); Dustin T. Libby, Naples, FL (US); Peter J. Dreyfuss, Naples, FL (US); James P. Bradley, Pittsburgh, PA (US)

(73) Assignee: Arthrex, Inc., Naples, FL (US)

(21) Appl. No.: 14/186,628

(22) Filed: Feb. 21, 2014

Related U.S. Application Data
(60) Provisional application No. 61/768,641, filed on Feb. 25, 2013.
(60) Provisional application No. 61/815,354, filed on Apr. 24, 2013.

(51) Int. Cl. A61B 17/04; A61L 17/00

(52) U.S. Cl. CPC A61B 17/0493; A61L 17/005; A61B 17/0401
USPC 606/230; 606/228; 606/231; 606/232

(57) ABSTRACT

Fixation of soft tissue to bone (or of soft tissue to soft tissue) is performed using a flexible material (for example, a suture strand, a braid, a suture tape, a stuffed suture, or a combination thereof) with a portion having an expanded footprint to provide a cushion or tissue protector between the flexible strand (suture) and the tissue to be attached. The suture and cushion may be manufactured from materials that have properties to amplify the body's healing response. The cushion may have any shape and geometry that provides cushioning action between the suture and the tissue to be fixated. The cushion may be provided along the length of the flexible strand (i.e., the flexible strand may be provided with a cushion) or the flexible strand may extend from the cushion.

### CROSS-REFERENCE TO RELATED APPLICATIONS

This application claims the benefit of U.S. Provisional Application Nos. 61/768,641, filed Feb. 25, 2013, and 61/815,354, filed Apr. 24, 2013, the disclosures of both of which are incorporated by reference in their entirety herein.

### FIELD OF THE INVENTION

The present invention relates to the field of surgery and, more particularly, to improved sutures and methods of tissue fixation.

### BACKGROUND OF THE INVENTION

Sutures have been known throughout the history of surgery. The variety of materials used to close wounds and attach soft tissue to bone (or soft tissue to soft tissue) has included wires of gold, silver, iron, and steel; dried gut; silk; animal hairs; tree bark and other plant fibers; and, more recently, a wide selection of synthetic compositions such as ultrahigh molecular weight polyethylene (UHMWPE), or the FiberWire® suture disclosed in U.S. Pat. No. 6,716,234 which is hereby incorporated by reference in its entirety.

Improved surgical sutures that would allow the user to use a preferred suture during a surgical procedure with the ability to widen the footprint where the suture will be in contact with tissue are needed to reduce the likelihood of suture tear through. Also needed are improved methods of surgical tying and suture manipulation with decreased suture tear, as well as methods of maximizing benefits of tissue cut-through resistance with minimal and efficient use of material.

### SUMMARY OF THE INVENTION

The present invention provides methods and tissue fixation and protection constructs including a flexible strand (for example, suture) for fixation of soft tissue to bone, or of soft tissue to soft tissue, which amplifies the body's healing response created by the introduction of the suture. Fixation of soft tissue to bone (or of soft tissue to soft tissue) is performed using a flexible material (for example, a suture strand, a braid, a suture tape, a stuffed suture, or a combination thereof) with a portion having an expanded footprint to provide a cushion or tissue protector between the flexible strand (suture) and the tissue to be attached. The suture and cushion may be manufactured from materials that have properties to amplify the body's healing response. The cushion may have any shape and geometry that provides cushioning action between the suture and the tissue to be fixated. The cushion may be provided along the length of the flexible strand (i.e., the flexible strand may be provided with a cushion) or the flexible strand may extend from the cushion.

These and other features and advantages of the present invention will become apparent from the following description of the invention that is provided in connection with the accompanying drawings and illustrated embodiments of the invention.

### BRIEF DESCRIPTION OF THE DRAWINGS

FIGS. 1 and 2 illustrate subsequent steps of a method of fixating soft tissue to bone (shoulder repair) with a suture construct formed of suture with a suture cushion (suture with cushion), and in accordance with an exemplary embodiment of the present invention.

FIG. 3 illustrates a perspective view of a suture construct according to a first embodiment of the present invention (with a flexible strand and a suture cushion in the shape of a bar provided with means for holding the cushion to the suture).

FIG. 4 illustrates a front view of the suture construct of FIG. 3.

FIG. 5 illustrates a schematic perspective view of the suture construct of FIG. 3 used for attachment of labrum to the glenoid.

FIG. 6 illustrates a perspective view of a suture construct according to a second embodiment of the present invention (with a flexible strand and a suture cushion in the shape of a hollow tubular member attached to the suture).

FIG. 7 illustrates a front view of the suture cushion of FIG. 6.

FIG. 8 illustrates a tissue protector suture construct in accordance with another exemplary embodiment of the present invention.

FIG. 9 illustrates the tissue protector suture construct of FIG. 8 employed for fixation of tissue to bone, and in accordance with an exemplary embodiment of the present invention.

FIG. 10 illustrates a tissue protector of a suture construct according to another exemplary embodiment of the present invention.

FIG. 11 illustrates a tissue protector suture construct in accordance with yet another exemplary embodiment of the present invention.

FIG. 12 illustrates the tissue protector suture construct of FIG. 11 employed for fixation of tissue to bone, and in accordance with an exemplary embodiment of the present invention.

FIGS. 13(a)-13(c) illustrate a tissue protector suture construct in accordance with yet another exemplary embodiment of the present invention.

FIG. 14 illustrates the tissue protector suture construct of FIG. 13(c) employed for fixation of tissue to bone, and in accordance with an exemplary embodiment of the present invention.

FIGS. 15 and 16 illustrate two tissue protector suture constructs in accordance with yet additional exemplary embodiments of the present invention.

### DETAILED DESCRIPTION OF THE PREFERRED EMBODIMENTS

The present invention provides methods and constructs for fixation of soft tissue to bone (or of soft tissue to soft tissue) which amplify the healing response created by the introduction of a suture material and the material properties of an attached suture cushion, resulting in the elimination of suture slippage and suture tear associated with the suture.

As detailed below, and according to an exemplary embodiment, fixation of soft tissue to bone is performed using a suture (for example, a suture strand, braid, suture tape, suture with collagen, stuffed suture, or a combination thereof) with a suture cushion arranged along a length of the flexible material, so that the flexible strand (suture) is cushioned against the tissue to be attached. The material properties of the suture cushion amplify the healing response when implanted/secured in vivo.

In additional exemplary embodiments, the present invention provides constructs for fixation of soft tissue to bone (or of soft tissue to soft tissue) which protect the soft tissue with an attached tissue protector, resulting in the elimination of suture slippage and suture tear associated with passing the suture around the soft tissue. The tissue protector suture construct may be formed of one or more flexible strands (sutures, braids, tapes, or combinations) spliced onto ends of a synthetic or biologic tissue protector (center component). Fixation of tissue to bone is achieved by using the construct passed around tissue and secured into bone with at least one fixation device, for example, at least one suture anchor.

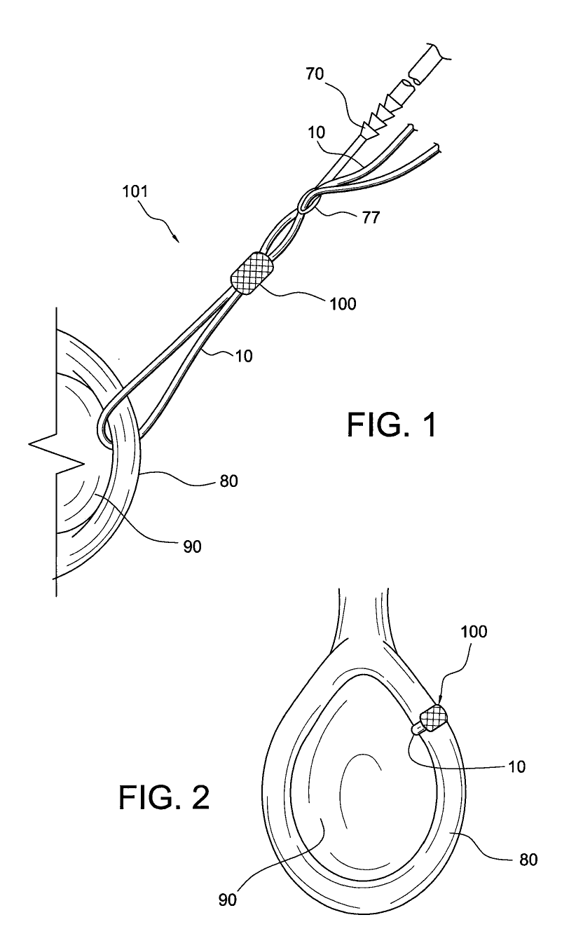

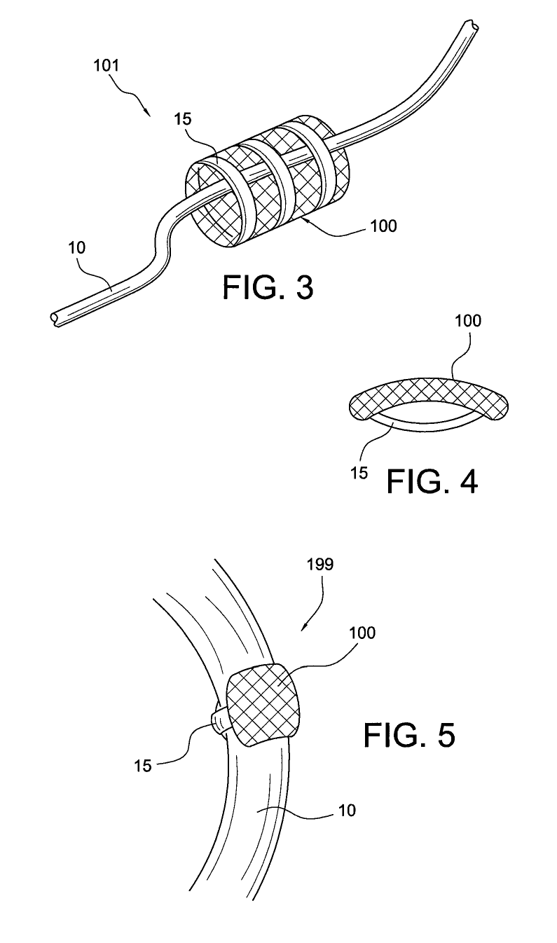

Referring now to the drawings, where like elements are designated by like reference numerals, FIGS. 1-16 illustrates exemplary tissue protector and fixation constructs 101, 101a, 201, 301, 401, 501, 601 of the present invention provided with at least one flexible material 10 (flexible strand 10) and a cushion 100, 100a, 200, 200a, 300, 400, 500, 600 arranged along (or extending from) a length of the at least one flexible material. The flexible strand may be removably attached to and/or detached from the cushion or may be provided integral to it. The cushion will be also referred below as to a tissue protector, or tissue protector and fixation construct, or a pad of material.

FIGS. 1-7

FIGS. 1-7 illustrates exemplary constructs 101, 101a of the present invention provided with a flexible material 10 (flexible strand 10) and a suture cushion 100, 100a arranged along a length of the flexible material.

The constructs 101, 101a are formed of a flexible material 10 (for example, a suture strand, a braid, a suture tape, or a combination thereof) with a portion 100, 100a having an expanded footprint to provide a cushion between the flexible strand (suture) and the tissue to be attached. The suture 10 and cushion 100, 100a may be manufactured from materials that have properties to amplify the body's healing response. For example, the cushion 100, 100a may be provided with a medicinal or therapeutic agent, for example, antiseptics, antibiotics, drugs, pharmaceutical agents, hormones and growth materials (for example, autogenous growth factors such as platelet-rich plasma (PRP), autologous factors, autologous-conditioned plasma (ACP)), among many others.

The cushion 100, 100a may have any shape and geometry that provides cushioning action between the suture and the tissue to be fixated. For example, and according to an exemplary-only embodiment, the cushion 100a is a generally elongated, hollow tubular structure (elongated cylinder or ring) that is securely attached to the flexible strand (suture) and allowed to slide along the length of the flexible strand. In yet another embodiment, the cushion 100 is a bar having a general rectangular or square configuration and provided with at least one connector to connect the cushion (the suture bar) to the flexible strand (suture) 10.

FIGS. 1 and 2 illustrate a schematic view of a surgical site undergoing a method of fixation of soft tissue to bone (or of soft tissue to soft tissue) by the methods of the present invention. In an exemplary embodiment only, the surgical site is the shoulder and the tissue is labrum 80 to be attached to glenoid 90 with constructs 101, 101a of the present invention. FIG. 1 also illustrates an exemplary knotless fixation device 70 with an eyelet 77 that allows ends of flexible strand 10 (suture 10) to pass therethrough and aid in the fixation of the labrum 80 to the glenoid 10.

The cushion 100, 100a may have any shape and geometry to provide increased cushioning action between the suture 10 and the tissue 80 to be fixated. For example, FIGS. 1-5 illustrate various views of a first suture construct 101 with a suture 10 and a suture cushion 100 according to a first embodiment of the present invention. Cushion 100 is a bar having a general rectangular or square configuration and provided with at least one connector 15 to connect the cushion (the bar) to the flexible strand (suture). The at least one connector 15 includes means for holding the cushion 100 to suture 10, for example, a plurality of attachment points that allow suture strands to pass therethrough and hold the cushion (bar) to the flexible strand 10.

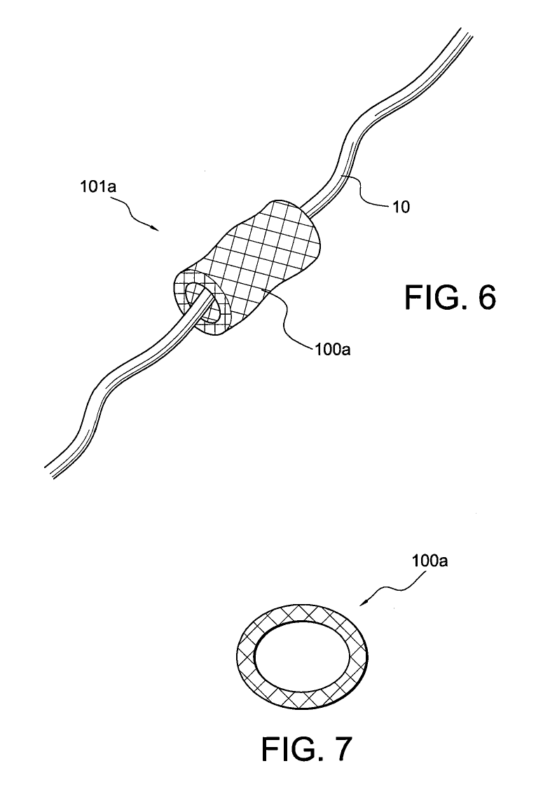

FIGS. 6 and 7 illustrate various views of suture construct 101a with flexible strand (suture) 10 and a cushion 100a according to a second exemplary embodiment of the present invention. According to this embodiment, cushion 100a is a generally elongated, hollow tubular structure (elongated cylinder or ring) that is securely attached to the flexible strand 10 (suture 10) and allowed to slide along the length of the flexible strand 10.

The suture 10 and cushion 100, 100a of constructs 101, 101a are preferably manufactured from materials that have properties to amplify the body's healing response.

In an exemplary and illustrative embodiment only, the flexible material 10 is a suture strand (for example, suture, or suture material such as ultrahigh molecular weight polyethylene (UHMWPE), or the FiberWire® suture) or a tape (for example, a collagen tape or a collagen stuffed suture) or any flexible material that allows suturing and/or fixation of tissue to tissue. The flexible material 10 (suture 10) may be provided with optional colored strands to assist surgeons in distinguishing between suture lengths with the trace and suture lengths without the trace. The flexible material 10 may also contain a bioabsorbable material, such as PLLA or one of the other polylactides, for example, and/or may be formed of twisted fibers having strands of a contrasting color added to the braided threads, to make the suture more visible during surgical procedures.

In an exemplary and illustrative embodiment only, the cushion 100, 200 may be formed of suture, suture-like material, foam, polymers, elastic materials, deformable, flexible or rigid materials, or any other material that is body compatible and provides less trauma and irritability to the tissue. The cushion 100, 200 may be optionally provided with a medicinal or therapeutic agent, for example, antiseptics, analgesics, antibiotics, drugs, pharmaceutical agents, hormones, diagnostic agents and growth materials (for example, autogenous growth factors such as platelet-rich plasma (PRP), autologous factors, autologous-conditioned plasma (ACP)), among many others.

The suture construct 101, 101a and cushion 100, 100a of the present invention may be employed in surgical procedures such as rotator cuff repair, Achilles tendon repair, patellar tendon repair, ACL/PCL reconstruction, hip and shoulder reconstruction procedures.

Fixation of soft tissue to bone, such as fixation of labrum 80 to glenoid 90, typically involves the formation of an incision to access the surgical site and then reattachment of the soft tissue. When soft tissue is attached to bone, the surgeon drills a cavity in the bone and inserts a fixation device 70 such as a bone anchor 70. Typically, the bone anchor 70 is formed of metal, composite, plastic or bioabsorbable material, and is held in place by threads or by barbs. If an anchor is employed (such as anchor 70), the anchor typically includes an eyelet 77 through which suture 10 with suture cushion 100, 100a is threaded.

After placing the anchor 70, the surgeon ties the suture 10 through the soft tissue, connecting it to the eyelet 77 of the bone anchor 70, thus re-approximating the soft tissue 80 to the bone 90. The technique is repeated multiple times at different locations in the bone. If multiple sutures are used, however, regrowth of the soft tissue during natural healing is difficult. By providing the cushion 100, 100a attached to the suture, both the footprint of the suture coming into contact with tissue and the healing of tissue 80 are increased, and suture cut through is minimized.

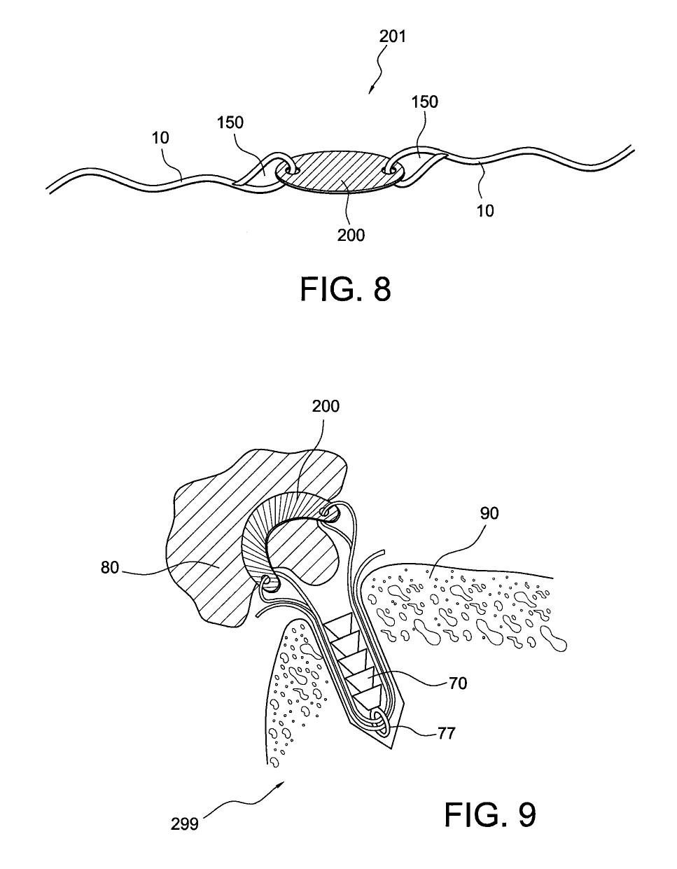

FIGS. 8-10

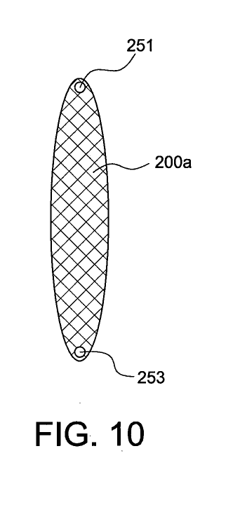

FIGS. 8-10 illustrate exemplary tissue protectors 200, 200a for attachment to flexible materials (flexible strands such as suture) to form the tissue protector suture construct 201. FIGS. 8 and 9 illustrate exemplary suture construct 201 provided with a plurality of flexible materials 10 (flexible strands 10) and a tissue protector 200 attached to the flexible materials. FIG. 10 illustrates another exemplary tissue protector 200a with two small eyelets, each provided at an opposite end of the construct 200a.

Tissue protector construct 201 may be formed of one or more flexible strands 10 (sutures, braids, tapes, or combinations) spliced onto ends of a synthetic or biologic tissue protector (center component) 200, 200a. Fixation of tissue to bone is achieved by using the construct passed around tissue and secured into bone with at least one fixation device, for example, at least one suture anchor.

The tissue protector 200, 200a is preferably wide, soft and strong, and made of various materials and in different shapes and geometries, to provide protection to the tissue to be fixated. The tissue protector 200, 200a may be manufactured from materials that have properties to amplify the body's healing response. For example, the tissue protector 200, 200a may be provided with a medicinal or therapeutic agent, for example, antiseptics, antibiotics, drugs, pharmaceutical agents, hormones and growth materials (for example, autogenous growth factors such as platelet-rich plasma (PRP), autologous factors, autologous-conditioned plasma (ACP)), among many others.

The tissue protector 200, 200a may have any shape and geometry that provides cushioning action to the tissue to be fixated. For example, and according to an exemplary-only embodiment, the tissue protector is a generally elongated structure with an oval or rectangular configuration, and also wide, soft and yet strong, and securely attached to one or more flexible strands (sutures). In yet another embodiment, the tissue protector 200, 200a is provided with at least one connector (for example, an eyesplice or eyesplice interconnection) to connect the tissue protector to the flexible strands (sutures). In yet other embodiments, the flexible strands (sutures) could just be tied to the tissue protector, without using a connector such as an eyesplice. Alternatively, the flexible strands (sutures) could be attached to the tissue protector by any other means of attachment known in the art.

FIG. 9 illustrates a schematic view of a surgical site undergoing a method of fixation of soft tissue to bone (or of soft tissue to soft tissue) by the methods of the present invention. In an exemplary embodiment only, the surgical site is the shoulder and the tissue is labrum 80 to be attached to glenoid 90 with construct 101 of the present invention. FIG. 9 also illustrates an exemplary knotless fixation device 70 with an eyelet 77 that allows ends of flexible strands 10 (sutures 10) to pass therethrough and aid in the fixation of the labrum 80 to the glenoid 90. The flexible strands may be also secured by employing two fixation devices, for example, two knotless fixation devices 70 with two eyelets 77, in lieu of the one fixation device.

FIG. 10 illustrates another exemplary tissue protector 200a of the present invention, according to which the tissue protector 200a is provided with two small eyelets 251, 253 at each of the ends of the body of the tissue protector 200a. The one or more flexible strands (sutures) may be simply passed through the ends with eyelets 251, 253 and then fixed with one fixation device (for example, one anchor) or, alternatively, with two fixation devices (for example, two anchors) spaced apart the length of the construct.

The tissue protector 200, 200a may have any shape and geometry that provide increased cushioning action to tissue 80 to be fixated. For example, tissue protector 200, 200a may be any structure that is soft, wide and strong to cushion the tissue to be fixated. Tissue protector 200, 200a may be formed of synthetic or biological material, may be braided or woven, formed of textiles and/or as a mesh or other configurations.

In an exemplary embodiment, tissue protector 200 is an elongated, oval structure as shown in FIG. 8 provided with at least one connector 150 for connecting the tissue protector 200 to the flexible strands (sutures). The at least one connector 150 includes means for holding the tissue protector 200 to sutures 10, for example, a plurality of eyesplices 150 or similar structures that allow suture strands 10 to securely attach to and hold the flexible strands 10 to the tissue protector 200. In the embodiment illustrated in FIG. 10, the flexible strands pass directly through the small eyelets 251, 253 provided at opposing ends of the tissue protector 200a and then fixed with one or more fixation devices (for example, one or more suture anchors).

The sutures 10 and tissue protector 200, 200a may be manufactured from materials that have properties to protect the soft tissue and amplify the body's healing response. In an exemplary and illustrative embodiment only, at least one of the flexible materials 10 is a suture strand or suture braid with a hollow core (for example, suture material such as ultrahigh molecular weight polyethylene (UHMWPE), or the FiberWire® suture) or a tape (for example, a collagen tape or a collagen stuffed suture) or any flexible material that allows splicing, suturing and/or fixation of tissue to tissue. The flexible material 10 (suture 10) may be provided with optional colored strands to assist surgeons in distinguishing between suture lengths with the trace and suture lengths without the trace. The flexible material 10 may also contain a bioabsorbable material, such as PLLA or one of the other polylactides, for example, and/or may be formed of twisted fibers having strands of a contrasting color added to the braided threads, to make the suture more visible during surgical procedures.

In an exemplary and illustrative embodiment only, the tissue protector 200, 200a may be formed of suture, suture-like material, foam, polymers, elastic materials, deformable, or flexible materials, synthetic materials, or any other material that is body compatible and provides less trauma and irritability to the tissue. The tissue protector 200, 200a may be optionally provided with a medicinal or therapeutic agent, for example, antiseptics, analgesics, antibiotics, drugs, pharmaceutical agents, hormones, diagnostic agents and growth materials (for example, autogenous growth factors such as platelet-rich plasma (PRP), autologous factors, autologous-conditioned plasma (ACP)), among many others.

The suture construct 201 and tissue protector 200, 200a of the present invention may be employed in surgical procedures such as rotator cuff repair, Achilles tendon repair, and patellar tendon repair, among many others.

As shown in FIG. 9, the surgeon places the tissue protector 200 around the soft tissue 80 and ties the sutures 10 through the soft tissue 80, connecting the sutures to the eyelet 77 of the bone anchor 70, thus re-approximating the soft tissue 80 to the bone 90 to achieve repair 299. The technique is repeated multiple times at different locations in the bone. By providing the tissue protector 200, 200a attached to the sutures, both the footprint of the suture coming into contact with tissue and the healing of tissue 80 are increased, and suture cut through is minimized.

In yet additional embodiments, each flexible strand (suture end) may be connected to a separate fixation device, for example, to two separate suture anchors spaced apart the length of the construct. Thus, the invention is not limited to the exemplary-only embodiment shown in FIG. 9 that employs only one fixation device, and has applicability to embodiments and constructs having the at least one flexible strand/material secured with two or more fixation devices. The tissue protector is cushioned against (protects) the tissue to be fixated/attached. The material properties of the tissue protector amplify the healing response when implanted/secured in vivo.

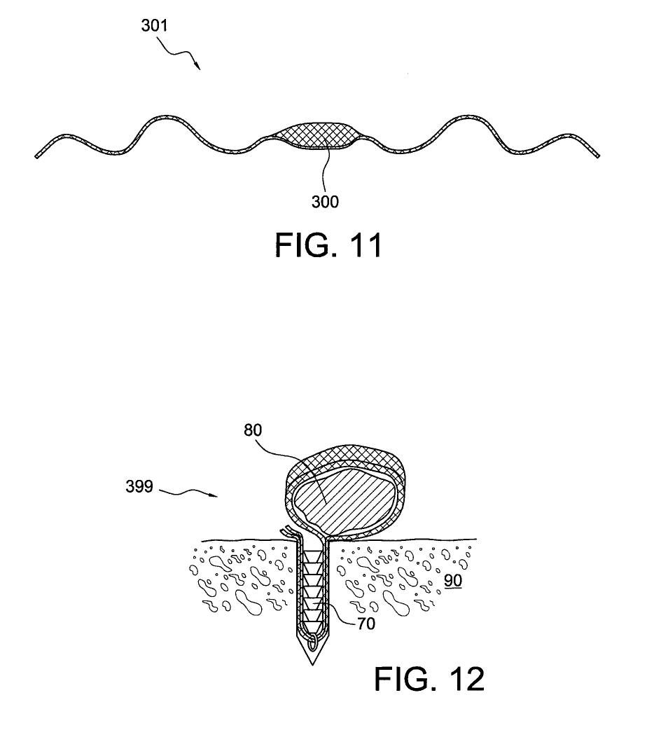

FIGS. 11 and 12

FIGS. 11 and 12 illustrate construct 301 which includes a wide flat tape portion 300 that is much shorter in length than FiberTape® (U.S. Pat. No. 7,892,256) with a coreless suture-like tail 10. The flat tape is provided about in the middle area and is flanked by two exemplary suture-like ends. Construct 301 allows a smaller anchor to be used in the repair because the tails are used to secure the construct with the fixation device (for example, PushLock® anchor, disclosed in U.S. Pat. No. 7,993,369) in the hole and they are much smaller than the FiberTape®. During repair 399 (FIG. 12), the broad flat tape is provided over the soft tissue 80 (over the tissue repair) and the suture ends are placed in a small pilot hole formed within bone 90 (and passed through the eyelet of the fixation device/knotless anchor 70).

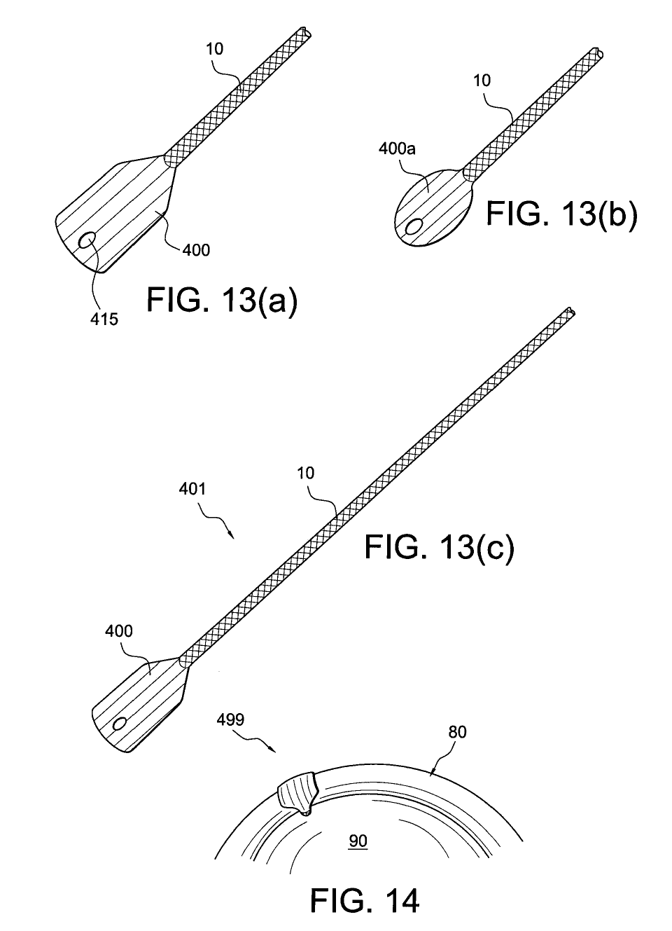

FIGS. 13(a)-14

Tissue protector construct 401 is formed of a braided or woven portion 400, 400a provided on one end of a flexible strand 10, for example, a standard round diameter suture 10. The construct has a wider suture footprint and is used to “cinch” or “tag” stitch the soft tissue 80 (for example, labrum 80).

Instead of one strand of suture splicing into itself, suture construct 401 has the braided/woven portion 400 on one end of the round diameter suture. In this “tape-like” portion of suture, there is a reinforced hole 451 (FIG. 13(a)) of which the standard suture will pass through after having passed through the soft tissue.

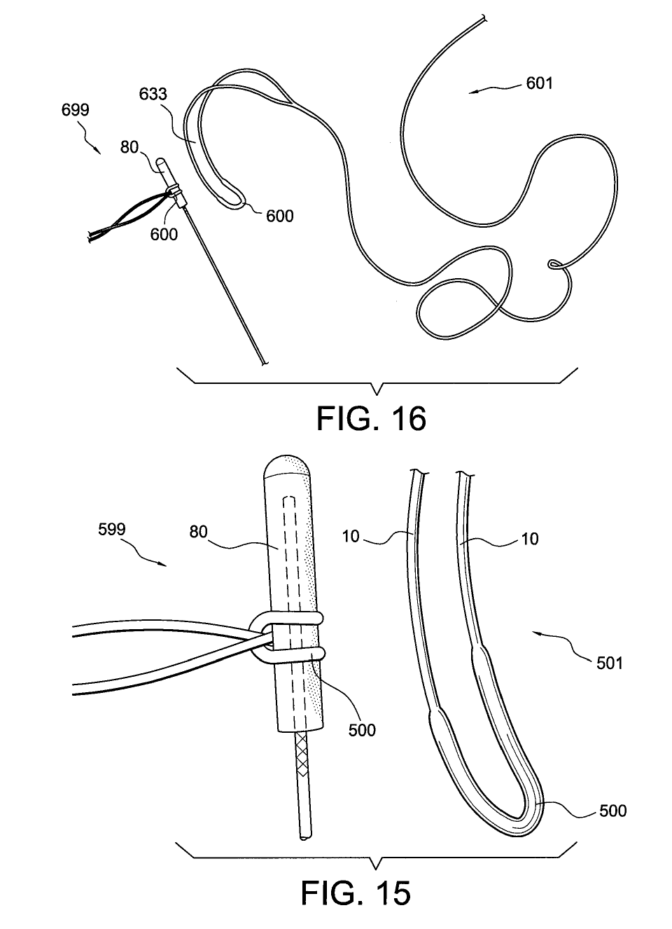

FIGS. 15 and 16

FIGS. 15 and 16 illustrate constructs 501, 601 which are about similar to the construct 301 of FIG. 11 but differ in that the flat tape portion 300 of the construct of FIG. 11 is replaced with a middle portion that is stuffed but not tape. FIG. 15 illustrates, for example, middle portion 500 which has a larger rounder diameter than the ends 10. During repair 599, the stuffed portion 500 is provided over exemplary soft tissue 80 (over the tissue repair) and the suture ends are placed in a hole formed within bone 90, as detailed above with reference to the previously-described embodiments.

FIG. 16 illustrates construct 601 which is about similar to the construct 501 of FIG. 15 but differs in that the ends 10 are joined to form flexible loop 633 (as in the FiberLink) and the center portion 600 of loop 633 is stuffed. During repair 699, the stuffed portion 600 is provided over exemplary soft tissue 80 (over the tissue repair) and the suture ends are placed in a hole formed within bone 90, as detailed above with reference to the previously-described embodiments.

Center portion 500, 600 of constructs 501, 601 is stuffed and has a length of about an inch long, centered on the end of the loop (for FIG. 16 construct). When a cinch is created around tissue 80, the stuffed (thicker) part 500, 600 is therefore always centered at the repair.

Although the invention has been described with reference to a particular application (i.e., fixation of labrum to glenoid in a shoulder repair), it must be understood that the construct of the present invention has applicability to any type of repairs (any repair in addition to a shoulder repair) and, thus, the invention is not limited by this exemplary-only embodiment.

Although the invention has been described with reference to only one cushion (body member, or pad, or tissue protector) provided along a length of flexible material, the present invention also contemplates embodiments wherein a plurality of cushions are provided along one or more flexible strands (sutures).

Fixation device 70 detailed above may be a knotless fixation device, for example, a knotless suture anchor such as the two-piece Arthrex PushLock® anchor, disclosed in U.S. Pat. No. 7,329,272, or an Arthrex SwiveLock® anchor, disclosed in U.S. Pat. No. 8,012,174 (the disclosures of which are herein incorporated by reference in their entireties). As detailed in these patents and applications, a knotless suture anchor is formed of an implant with a distal eyelet (which may be closed or forked, and which engages and secures at least one flexible strand) and a fixation device (a cannulated screw) that is advanced over the implant body to secure the implant and the flexible strand into a bone hole or socket, and to approximate soft tissue to bone. The implant may be rotatably attached to (and/or may swivel relative to) a tip of a driver assembly that is pre-loaded with the separate, cannulated fixation device (cannulated screw).

While the present invention is described herein with reference to illustrative embodiments for particular applications, it should be understood that the invention is not limited thereto. Those having ordinary skill in the art and access to the teachings provided herein will recognize additional modifications, applications, embodiments and substitution of equivalents all fall within the scope of the invention. Accordingly, the invention is not to be considered as limited by the foregoing description.

What is claimed is:

1. A tissue protector construct, comprising:
at least one flexible strand; and
a cushion attached to the at least one flexible strand.

2. The tissue protector of claim 1, wherein the cushion is removably attached to the at least one flexible strand.

3. The tissue protector of claim 1, wherein the cushion is permanently affixed to the at least one flexible strand.

4. The tissue protector of claim 1, wherein the cushion is provided along a length of the at least one flexible strand and supported by the at least one flexible strand.

5. The tissue protector of claim 1, wherein the at least one flexible strand is suture and the cushion slides along the suture.

6. The tissue protector of claim 1, wherein the cushion comprises a plurality of eyelets, and wherein the at least one flexible strand attaches to the eyelets.

7. The tissue protector of claim 1, wherein the cushion is formed of a material selected from the group consisting of yarns, filaments, fibers, suture strands, textiles, suture tape, polymers, elastic materials, foam and suture-like materials.

8. The tissue protector of claim 1, wherein the cushion further comprises a biological component selected from the group consisting of platelet-rich plasma, autologous conditioned plasma and bone marrow aspirate.

9. The tissue protector of claim 1, wherein the cushion is a flat tape or a stuffed suture.

10. A tissue protector consisting essentially of a pad of material provided along a length of a material strand.

11. The tissue protector of claim 10, wherein the pad is in the form of a rectangle, a hollow cylinder, a square or an oval.

12. The tissue protector of claim 10, wherein the pad is provided along and around the length of material strand.

13. The tissue protector of claim 10, wherein the pad is formed of resorbable or non-resorbable material.

14. The tissue protector of claim 10, wherein the material strand is a suture, a nitinol strand, a yarn, a filament, a fiber strand, a suture tape, a collagen tape or a suture chain.

15. The tissue protector of claim 10, wherein the pad is provided with at least an eyelet.

16. A tissue protection and fixation construct consisting essentially of a flexible strand with a cushion attached to the flexible strand, and a knotless fixation device provided with a closed eyelet at its most distal end to allow the flexible strand to pass thereto and to anchor the flexible strand into bone.

17. The construct of claim 16, wherein the knotless fixation device is a swivel anchor.

18. A method of fixation of a first tissue to a second tissue, comprising the steps of:
providing a suture tissue protector comprising a length of a flexible strand and at least one cushion provided along the length of the flexible strand, the cushion consisting essentially of suture or suture-like materials;
passing the flexible strand around the first tissue so that the cushion is positioned over and in contact with the first tissue;
attaching the flexible strand to a knotless fixation device; and
placing the knotless fixation device with the attached flexible strand into the second tissue.

19. The method of claim 18, wherein the first tissue is soft tissue and the second tissue is bone.

20. The method of claim 18, wherein the flexible strand is one of a suture strand, a suture tape, a stuffed suture, a suture chain or a nitinol strand.

21. The method of claim 18, further comprising the step of providing a biological material to the cushion.

22. The method of claim 21, wherein the biological material is selected from the group consisting of blood, blood components, bone marrow aspirate, platelet rich plasma, autologous conditioned plasma, proteins, growth factors and hormones.

23. The method of claim 18, wherein the step of attaching the flexible strand to the knotless fixation device comprises threading the flexible strand through an eyelet of the knotless fixation device.
